(19) United States
(12) Patent Application Publication
(10) Pub. No.: US 2010/0221207 A1
(43) Pub. Date: Sep. 2, 2010

(54) FRAGRANCE COATED SALT CRYSTALS

(75) Inventors: Cheryl Watkins, Scottsdale, AZ (US); Tyler Mikkelsen, Scottsdale, AZ (US); Geoffrey Faires, Cave Creek, AZ (US)

Correspondence Address: THE DIAL CORPORATION, 19001 N. Scottsdale Road, SCOTTSDALE, AZ, 85255, US

(73) Assignee: The Dial Corporation, Scottsdale, AZ (US)

(21) Appl. No.: 12/503,231

(22) Filed: Jul. 15, 2009

Related U.S. Application Data
(60) Provisional application No. 61/156,581, filed on Mar. 2, 2009.

(51) Int. Cl. A61L 9/01; A61L 101/02

(52) U.S. Cl. 424/76.4

(57) ABSTRACT

An air freshener is described that comprises salt crystals coated with a premix comprising fumed silica, a nonionic emulsifier and a fragrance oil.

### PRIORITY APPLICATION

The present application claims priority to U.S. Provisional Application 61/156,581 filed Mar. 2, 2009 and entitled “FRAGRANCED SALT CRYSTAL AIR FRESHENER AND KIT FOR ASSEMBLING SAME”, which is incorporated herein.

### FIELD OF INVENTION

The present invention relates generally to air fresheners and in particular to a passive air freshener comprising fragrance coated salt crystals grown from brine evaporation, having improved fragrance delivery and visual aesthetics.

### BACKGROUND

Air fresheners have been in the marketplace and in the patent literature for many decades. Consumer air fresheners run the gamut from static blocks of fragranced gel to programmable and playable electronic devices with heaters and/or fans, and correspondingly are sold across a wide price range. Other products are simple aerosols, such as the Renuzit® Subtle Effects™ aerosol sprays. “Passive” refers to air fresheners that do not require any energy to operate. That is, a passive air freshener can scent a room simply sitting at ambient temperature in a home. These passive air freshener products release volatile fragrance components from a solid matrix, pad, or other carrier over time, with the evaporation rate (and product length of life) usually determined by the volatility of the fragrance mixture, the surface area from which the fragrance mixture can evaporate, and the extent to which airflow can interact with the product. Some passive air fresheners rely on emanating pads or wicks having very large surface areas to evaporate a volatile liquid fragrance mixture into the room. Other typical passive air fresheners are static blocks of fragranced gel, such as the Renuzit® Adjustable Cone air freshener that has been in the market for many years.

Other examples of passive air fresheners include: fragranced salt granules (loose or in vapor-permeable sachets); fragranced mineral rocks or mined crystalline material; and, molded, tableted or pelletized salts admixed with fragrances. These variations are described in a number of exemplary references including: U.S. Pat. No. 3,371,984 (Kelly, et al.); U.S. Pat. Nos. 5,041,421 and 5,246,919, and PCT Application Publication WO 90/04960 (King); U.S. Pat. No. 6,270,720 (Mandish); U.S. Pat. No. 6,426,325 (Dente, et al.); and, U.S. Patent Application Publication 2006/0293215 (Sizemore). The King references (U.S. '421 and '919, and WO 90/04960) describe a variety of fragranced salt products, including fragranced salt pellets and molded/tabletized material formed from compression of scented salt granules in a tablet press. In King's first embodiment, ('919, Col. 5, EXAMPLE 1) porous salt pellets (compressed pellets sold by Morton® Salt for water softeners) were sprayed with a mixture of fragrance oil and dye in a rotating drum mixer. In the other embodiments, ('919 EXAMPLES 2-4), King embeds a sufficient amount of slow-releasing fragrance within a finished tablet or molded shape by first coating the smaller granulated material with a mixture of fragrance and binder/desiccant (e.g. Cab-O-Sil), and then compressing the coated granulate into larger pellets, tablets or shapes. This method is limiting as it produces only opaque uniform shapes, rather than utilizing the natural beauty and variation found in naturally grown crystals. Mandish ('720) claims a salt sculpture air freshener comprising a piece of sculpted halite coated with a mixture of fragrance and “dry cement”, but the composition of the coating is not described. Clearly, these references do not adequately teach how to effectively coat fragrance onto individual large salt crystals (such as those grown through evaporation of sea water or other salt/brine solution) in order to optimize static fragrance delivery from such coated crystals. Indeed, since salt crystals grown through evaporation of brine have minimal porosity, it's not likely that fragrance oils can absorb into the crystals. As an example of this teaching, Sizemore (in Application 2006/0293215) states that salt crystals are not porous to fragrance oils (PARA [0003]) and consequently discloses an idea to use electromagnetic radiation to improve the coating of salt crystals.

Examples of salt crystal air fresheners for purchase may be found on the Internet under the general description “crystal potpourri” or “crystal rock potpourri”. These products generally appear to be both semi-porous mineral or sea salt rocks. Although some Internet sites suggest a home recipe to coat the salt crystals with a mixture of food coloring, essential fragrance oils and glycerin, or to simply shake the crystals in a plastic bag with fragrance oil to restore the scent on previously fragranced rocks, there is no disclosure of how best to coat salt crystals to make long lasting and efficacious air fresheners.

Therefore what is clearing lacking in the literature is an effective passive air freshener comprised of larger natural salt crystals grown through evaporative methods, coated with an efficacious level of fragrance such that the crystals retain their aesthetic beauty yet are able to deliver consumer-acceptable fragrance levels over a reasonable length of time.

### SUMMARY OF THE INVENTION

It has now been discovered that tumble-coating large, natural, evaporative-sourced salt crystals of a specific size range with a pre-blend comprising fragrance oil, fumed silica, and a nonionic emulsifier results in air freshener crystals of remarkable appearance and fragrance intensity. That being said, the present invention is an air freshener comprising: natural, evaporative-sourced salt crystals sieved to range in size from about ⅜ inch to about 1 inch; fragrance oil; fumed silica; and, a nonionic emulsifier, with optional dyes, preservatives and other adjuvant. The size range of the fragranced salt crystals has been found to be critical for maximizing the fragrance perception released from an open jar of the crystals.

### DETAILED DESCRIPTION OF THE INVENTION

The following description is of exemplary embodiments only and is not intended to limit the scope, applicability or configuration of the invention in any way. Rather, the following description provides a convenient illustration for implementing exemplary embodiments of the invention. Various changes to the described embodiments may be made in the relative amounts of the ingredients described without departing from the scope of the invention as set forth in the appended claims. Most importantly, changes in shape of the crystals, as possibly achieved by switching between various methods for evaporation of the brine (solar, ambient, heat, or combinations thereof), or the source for the brine (sea water, Great Salt Lake water, brine wells, or manufactured saturated salt water solutions) are within the scope of the invention. Although the present invention is described in utilitarian terms as fragranced “natural salt” crystals, no restriction as to the chemical makeup of the crystals, or the source of the starting brine, is herein implied. For example, the salt may be comprised of potassium ions, mixed sodium and potassium ions, or mostly sodium ions, provided that the salt is comprised essentially of all chloride salts. The only limitation to the scope of the invention is that the crystals used herein are grown through evaporation of a salt solution, (be it ocean water, salt lake water, brine well water, or manufactured saturated salt solutions). That is, the present invention comprises “evaporative-sourced” crystals and does not include mined salt crystals such as halite, (i.e. rock salt). Most preferred is to use larger natural sea salt crystals as obtained through evaporation of natural sea water or even Great Salt Lake water, (via solar, wind, ambient air or heated methods of evaporation).

That said, the present invention relates to a passive air freshener comprising evaporation-sourced salt crystals coated with a pre-blend comprising fumed silica, fragrance oil, and emulsifier and optionally, dyes. More specifically the present invention is an air freshener composition minimally comprising: (a) evaporative-sourced salt crystals with size distribution falling between about ⅜ inch and about 1 inch sieve sizes; (b) fragrance oil; (c) fumed silica; and (d) a nonionic emulsifier. Optionally, the present invention may include one or more dyes or other suitable colorants, and preservatives (e.g. uv stabilizers, antioxidants, mold inhibitors, etc.). The method of manufacture for the present invention comprises the pre-mixing of the fumed silica with the fragrance, next adding the emulsifier (and optional dyes) and then using this fragrance premix to coat the salt crystals. The method of use of the present invention is to place a plurality of the said fragranced crystals into an open container and place the container at ambient conditions in the area to be scented (e.g. a room in a home).

Evaporative-Source Salt Crystals

Salt crystals suitable for use in the present air freshener comprise crystals that are obtained through evaporation of a salt/brine solution comprised primarily of sodium chloride (NaCl), potassium chloride (KCl) or mixed salt brine. Herein “brine” is taken to be a chloride salt solution, (i.e. M⁺Cl⁻, where M⁺ is Na⁺, K⁺, Mg²⁺, etc.). Preferably the crystals are simply natural sea or lake salt crystals that are obtained by evaporation of Atlantic, Pacific or Caribbean ocean waters, or Great Salt Lake water, through various well known methods (e.g. solar, ambient air, wind, heat, or combinations of evaporative methods, some depending on weather). Sea salt crystals are available from many suppliers such as Cargill, Morton, ESSA (Exportadora de Sal) or Diamond, and most preferred is to use “solar salt” obtainable from Cargill (Caribbean) or ESSA (Pacific). These sea salt crystals are grown in open ponds of sea water exposed to the sun. Also of use in the present invention are salt crystals grown from evaporation of brine well water. Additionally, ordinary salt (e.g. powdered or granulated NaCl, KCl, or mixed salts) could be dissolved in water to form a saturated solution and that solution could then be used to grow crystals useful for the present invention. Regardless of the source of the brine to evaporate, the salt crystals for use in the present invention preferably range in size from about ⅜ inch to about 1 inch long, as obtained by subsequent sieving of salt crystals with ⅜ inch and 1 inch U.S. Standard sieves. It is advantageous to have crystals that give a high yield of this size range when sieved, (i.e. a minimum of both “fines” and huge “rock-like” crystals). The crystals to be scented herein are not mined minerals (i.e. not rock salt or halite). Nonetheless, crystal shapes from evaporation of brine vary considerably, and this natural distribution of shapes and sizes is desirable for this air freshener. In fact, solar salt crystals grown from ocean waters have variability simply because of the daily weather variation (hot sunny days, cold days, windy days, etc.) and the influence of other minerals that affect crystal growth, number of layers and clarity.

As mentioned, a distribution of crystal size is preferably used herein, and that distribution is obtainable by collecting the crystals that remain trapped between stacked 1 inch and ⅜ inch U.S. Standard sieves. The remains on top of the 1 inch sieve (huge “rock-like” crystals), and what falls all the way through both sieves (the “fines”), is discarded. Of course, unusually long and narrow shaped crystals may manage to slip through the 1 inch sieve, and those narrow crystals may have lengths of up to 2 to 4 inches. These rare crystals are still useful for the present invention because they tend to break up into smaller crystals during the fragrance coating process (i.e. during tumbling). As mentioned, the crystals trapped in between these two U.S. Standard sieves are ideal for the present invention. This “cut” ranges in size, shape, color, and clarity, giving uniqueness to the air freshener product. Since it is difficult to describe the shapes of the natural crystals, and equally difficult to measure across any one of many dimensions of any single crystal or plurality of crystals, the preferred size range for use herein is necessarily defined as a sieve range.

Using crystals that measure less than about ⅜ inch (i.e. crystals that pass through both sieves) will lead to fragranced crystals that pack too uniformly and tightly when placed in an open jar. When the fragranced crystals pack tightly there is insufficient air flow between the crystals and an unacceptable fragrance delivery results. The preferred sieved size range of from about ⅜ inch to about 1 inch, with some distribution in both size and shape, ensure that the fragranced decorative crystals will produce a consumer acceptable fragrance intensity when placed in an open jar. Thus, for the present invention, it is most preferred to use natural sea salt (e.g. “solar salt”) having a size distribution falling between about ⅜ inch and about 1 inch sieve sizes. Or in other words, it is most preferred to use a sieved “cut” of natural solar sea salt crystals that remain between stacked ⅜ inch and 1 inch U.S. Standard sieves, (i.e. crystals that has pass through the 1 inch screen but are stopped by the ⅜ inch screen).

Fumed Silica

Commercially available fumed silica (or silica fume) may be formed from the conversion of silicon tetrachloride with hydrogen and oxygen in the gas phase (e.g. flame process). Commercial fumed silica is sub-micron (e.g. nanometer), very pure silicon dioxide (SiO₂). It may be purchased under the trade names Aerosil® by Evonik-Degussa and Cab-O-Sil® from Cabot Corp. Particularly useful for the fragrance pre-mix described herein is the “untreated” or hydrophilic fumed silica such as Cab-O-Sil® M-5 or Aerosil® 200 (both general purpose untreated fumed silica having moderate surface area of about 200 m²/gram). Fumed silica traditionally finds use in liquids for rheology control, suspension, moisture resistance, viscosity stability and emulsification. For pre-blends herein comprising fragrance oil, nonionic emulsifier and fumed silica, the silica may function in most if not in all of these capacities. The fragrance pre-blend preferably comprises from about 0.1% to about 10% by weight fumed silica. Most preferred is to prepare a pre-blend comprising from about 0.1% to about 5% by weight fumed silica. The air freshener of the present invention (the finished salt crystals coated with a fragrance pre-blend) preferably comprises from about 0.001% to about 2% by weight fumed silica. Most preferred is to have the fumed silica at a level of from about 0.05% to 0.15% by weight of finished scented salt crystals.

Nonionic Emulsifier

The nonionic emulsifier for use in the present invention may comprise at least one nonionic material including: sorbitan esters; alkoxylated sorbitan esters; C₂-C₆ glycols; glycol esters; glycerin; glyceryl esters; alkoxylated glyceryl esters; amide waxes; fatty alcohols; monoalcohol esters; polyethylene glycol, polyethylene glycol esters; polypropylene glycol, polypropylene glycol esters, fatty alcohol alkoxylates; alkyl phenol alkoxylates; alkoxylated fatty acid esters; and other nonionic materials of surfactant classification (e.g. alkanolamides, amine N-oxides, alkylpolyglycosides, etc.), and mixtures thereof. Regardless of the nature of the nonionic material(s), it is preferred to use a total amount of nonionic emulsifier in the fragrance premix at from about 0.1% to about 5% by weight, and at from about 0.001% to about 2% of the total weight of the finished fragranced salt crystals once the premix is used to coat the crystals.

Preferred nonionic emulsifiers for use herein include the sorbitan derivatives such as the Span®, Brij®, Tween® and Atlas® products available from Croda (formerly Uniqema). These materials arc sorbitan esters generally comprising a fatty acid chain, the sorbitan linkage, and optionally an alkoxylate (e.g. polyoxyethylene, also termed “PEG”, or “EO”) chain. The more preferred nonionic emulsifier for use in the present invention includes the sorbitan esters, in particular 3-80 mole ethoxylated mono-, di-, or tri-fatty acid esters of sorbitan. These materials are available under the trade name of Tween® and Atlas® from Croda and include: polyoxyethylene (2) sorbitan monolaurate (Tween® 20); polyoxyethylene (4) sorbitan monolaurate (Tween® 21); polyoxyethylene (20) sorbitan monopalmitate (Tween® 40); polyoxyethylene (20) sorbitan monostearate (Tween® 60); polyoxyethylene (4) sorbitan monostearate (Tween® 61); polyoxyethylene (20) sorbitan tristearate (Tween® 65); polyoxyethylene (5) sorbitan monooleate (Tween® 81); polyoxyethylene (20) sorbitan monooleate (Tween® 80); polyoxyethylene (20) sorbitan trioleate (Tween® 85); and, polyoxyethylene (80) sorbitan monolaurate (Atlas® G-4280), and mixtures thereof. The sorbitan esters (i.e., non-alkoxylated) are also useful, and are available under the trade name Span® from Croda. These preferred nonionic materials include sorbitan monstearate (Span® 60); and, sorbitan tristearate (Span® 65). Most preferred is to incorporate Tween® 20, Tween® 60 and/or Tween® 80, or mixtures thereof into the fragrance premix at from about 0.1% to about 5% by weight, and at from about 0.001% to about 2% of the total weight of the finished fragranced salt crystals.

Other preferred nonionic emulsifiers for use herein include surfactants such as ethoxylated (EO), propoxylated (PO), or mixed ethoxylated/propoxylated (EO/PO) alkylphenol ethers; EO, PO or EO/PO C₄-C₁₆ fatty alcohols; EO, PO or EO/PO mono- and di-esters of aliphatic C₄-C₁₆ carboxylic acids; EO, PO or EO/PO branched aliphatic alcohols with a main aliphatic carbon chains of C₄-C₁₆; and, EO, PO or EO/PO hydrogenated castor oils (such as the Cremophor® materials from BASF). Preferred ethoxylated aliphatic alcohols for use in the present invention are available under the trade name Tomadol® from Tomah. Also preferred is Eumulgin® HPS from Cognis, which is a mixture of ethoxylated alcohols, EO/PO glycol ethers, and ethoxylated hydrogenated castor oil, along with the Genapol® products from Clariant. Other preferred nonionic surfactants include the amine oxide surfactants. The preferred amine oxide surfactant for use in the present invention is typically a trialkyl amine oxide, most preferably an alkyldimethylamine oxide. Examples of such materials that find use in the fragrance premix herein include Ammonyx® LO from Stepan, Barlox® 12 from Lonza Corporation, and Surfox® LO Special from Surfactants, Inc. Any of these nonionic materials or mixtures thereof may be incorporated into the fragrance premix at from about 0.1% to about 5% by weight, and at from about 0.001% to about 2% of the total weight of the finished fragranced salt crystals

Other preferred nonionic materials for use in the present invention include amide type nonionic surfactants, for example alkanolamides that are condensates of fatty acids with alkanolamines such as monoethanolamine (MEA), diethanolamine (DEA) and monoisopropanolamine (MIPA. Useful alkanolamides to assist in constructing a stable fragrance premix for use herein include ethanolamides and/or isopropanolamides such as monoethanolamides, diethanolamides and isopropanolamides in which the fatty acid acyl radical typically contains from 8 to 18 carbon atoms. Especially satisfactory are mono- and diethanolamides such as those derived from coconut oil mixed fatty acids or special fractions containing, for instance, predominately C₁₂ to C₁₄ fatty acids. Of particular use in this invention are mono- and diethanolamides derived from coconut oil mixed fatty acids, (predominately C₁₂ to C₁₄ fatty acids), such as those available from McIntyre Group Limited under the brand name Mackamide®. Most preferred is Mackamide® CMA, which is coconut monoethanolamide available from McIntyre. Amide surfactants, when used as the nonionic emulsifier or as a co-emulsifier in a mixture of emulsifiers, are incorporated into the fragrance premix at from about 0.1% to about 5% by weight, and at from about 0.001% to about 2% of the total weight of the finished fragranced salt crystals.

The fragrance premix applied to the salt crystals may also comprise alkyl polyglycoside surfactant as the nonionic component. The alkyl polyglycosides (APGs) also called alkyl polyglucosides if the saccharide moiety is glucose, are naturally derived nonionic surfactants. The alkyl polyglycosides that may be used in the present invention are fatty ester derivatives of saccharides or polysaccharides that are formed when a carbohydrate is reacted under acidic condition with a fatty alcohol through condensation polymerization. The APGs are typically derived from corn-based carbohydrates and fatty alcohols from natural oils in animals, coconuts and palm kernels. The alkyl polyglycosides that are preferred for use in the present invention contain a hydrophilic group derived from carbohydrates and is composed of one or more anhydroglucose units. Each of the glucose units can have two ether oxygen atoms and three hydroxyl groups, along with a terminal hydroxyl group, which together impart water solubility to the glycoside. The presence of the alkyl carbon chain leads to the hydrophobic tail to the molecule. When carbohydrate molecules react with fatty alcohol compounds, alkyl polyglycoside molecules are formed having single or multiple anhydroglucose units, which are termed monoglycosides and polyglycosides, respectively. The final alkyl polyglycoside product typically has a distribution of varying concentration of glucose units (or degree of polymerization). The APGs that may be used in the fragrance premix as the nonionic emulsifier component preferably comprise saccharide or polysaccharide groups (i.e., mono-, di-, tri-, etc. saccharides) of hexose or pentose, and a fatty aliphatic group having 6 to 20 carbon atoms. Preferred alkyl polyglycosides that can be used according to the present invention are represented by the general formula, Gₓ-O—R¹, wherein G is a moiety derived from reducing saccharide containing 5 or 6 carbon atoms, e.g., pentose or hexose; R¹ is fatty alkyl group containing 6 to 20 carbon atoms; and x is the degree of polymerization of the polyglycoside, representing the number of monosaccharide repeating units in the polyglycoside. Generally, x is an integer on the basis of individual molecules, but because there are statistical variations in the manufacturing process for APGs, x may be a non-integer on an average basis when referred to APG used as an ingredient for the detergent composition of the present invention. For the APGs of use herein, x preferably has a value of less than 2.5, and more preferably is between 1 and 2. Exemplary saccharides from which G can be derived are glucose, fructose, mannose, galactose, talose, gulose, allose, altrose, idose, arabinose, xylose, lyxose and ribose. Because of the ready availability of glucose, glucose is preferred in polyglycosides. The fatty alkyl group is preferably saturated, although unsaturated fatty chains may be used. Generally, the commercially available polyglycosides have C₈ to C₁₆ alkyl chains and an average degree of polymerization of from 1.4 to 1.6. APG surfactants, when used as the nonionic emulsifier or as a co-emulsifier in a mixture of nonionic materials, may be incorporated into the fragrance premix at from about 0.1% to about 5% by weight, and at from about 0.001% to about 2% of the total weight of the finished fragranced salt crystals.

The fragrance premix may also utilize polyether materials, such as a polyethylene or polypropylene glycol, or mixtures of these as the nonionic emulsifier. One such polyether useful in the fragrance premix is polyethylene glycol (or “PEG”). These materials are most readily obtained from the Dow Chemical Company under the brand name Carbowax®. Esters of PEG may also find use in the present invention. Non-limiting examples include: PEG (40) stearate; PEG (200) cocoate; PEG (200) monooleate; PEG (300) monooleate; PEG (300) monostearate; PEG (400) cocoate; PEG (400) dilaurate; PEG (400) diooleate; PEG (400) monolaurate; PEG (400) monooleate; PEG (400) monostearate; PEG (400) ricinoleate; PEG (600) dioleate; and, PEG (600) monolaurate. The fragrance premix may also utilize small molecular weigh glycols (i.e. C₂-C₆) such as ethylene glycol, propylene glycol, diethylene glycol or dipropylene glycol. Additionally, esters of these lower molecular weight glycols find use in the present invention. Some non-limiting examples include: diethylene glycol distearate; diethylene glycol monostearate; ethylene glycol monostearate; propylene glycol dioleate; propylene glycol monostearate; and, propylene glycol tricapryl caprate. Any of these glycols, glycol ethers, polyethers, and/or esters, when used as the nonionic emulsifier or as a co-emulsifier in a mixture of nonionic materials, may be incorporated into the fragrance premix at from about 0.1% to about 5% by weight, and at from about 0.001% to about 2% of the total weight of the finished fragranced salt crystals.

Additionally, monoalcohol esters find use in the present invention to emulsify the fragrance premix. These materials include: 2-ethylhexyl oleate; 2-ethylhexyl palmitate; 2-ethylhexyl tallowate; 2-ethylhexyl stearate; butyl oleate; butyl stearate; cetyl palmitate; cetyl stearate; decyl oleate; isocetyl isostearate; isocetyl stearate; isopropyl myristate; isopropyl oleate; isopropyl palmitate; isopropyl palmitate-stearate; isotridecyl stearate; isodecyl stearate; myristyl myristate; and, octyl palmitate. These alcohol esters, when used as the nonionic emulsifier or as a co-emulsifier in a mixture of nonionic materials, may be incorporated into the fragrance premix at from about 0.1% to about 5% by weight, and at from about 0.001% to about 2% of the total weight of the finished fragranced salt crystals.

Lastly, glycerin, glyceryl fatty acid mono-, di-, and tri-esters, and alkoxylated fatty acid glyceryl mono-esters may be used as the nonionic emulsifier herein, either alone or mixed with other nonionic materials discussed. These well known emulsifiers include such compounds as: glyceryl monostearate, monooleate, monopalmitate, monococoate, monotallowate, monomyristate, monoricinolate and the like; polyoxyethylene-glyceryl monostearate, monooleate, monopalmitate, monococoate, monotallowate, monomyristate, monoricinoleate, and the like, where the degree of ethoxylation is from about 7 to about 80; glyceryl di-stearate, -oleate, -palmitate, -cocoate, -tallowate, -myristate, -ricinolate, and the like; and, glyceryl tri-acetate, -stearate, -oleate, -palmitate, -cocoate, -tallowate, -myristate, -ricinolate, and the like. Glycerin and these glycerin derivatives, when used as the nonionic emulsifier or as a co-emulsifier in a mixture of nonionic materials, may be incorporated into the fragrance premix at from about 0.1% to about 5% by weight, and at from about 0.001% to about 2% of the total weight of the finished fragranced salt crystals.

It should be noted that depending on molecular weight and structure, some of these nonionic materials may be solid at room temperature. In that case, the fragrance premix may be heated and the liquefied premix may be coated onto the salt crystals.

Fragrance Oil

The fragrance oil used in the premix to be coated on the salt crystals may comprise one of more volatile organic compounds available from any of the now known, or hereafter established, perfumery suppliers, such as International Flavors and Fragrances (IFF) of New Jersey, Givaudan of New Jersey, Firmenich of New Jersey, etc. Many types of fragrances can be used in the present invention. Preferably the fragrance materials are volatile essential oils. The fragrances, however, may be synthetically derived materials (aldehydes, ketones, esters, etc.), naturally derived oils, or mixtures thereof. Naturally derived fragrance substances include, but are not limited to, musk, civet, ambergis, castoreum and like animal perfumes; abies oil, ajowan oil, almond oil, ambrette seed absolute, angelic root oil, anise oil, basil oil, bay oil, benzoin resinoid, bergamot oil, birch oil, bois de rose oil, broom abs., cajeput oil, cananga oil, capsicum oil, caraway oil, cardamon oil, carrot seed oil, cassia oil, cedar leaf, cedarwood oil, celery seed oil, cinnamon bark oil, citronella oil, clary sage oil, clove oil, cognac oil, coriander oil, cubeb oil, cumin oil, camphor oil, dill oil, estragon oil, eucalyptus oil, fennel sweet oil, galbanum res., garlic oil, geranium oil, ginger oil, grapefruit oil, hop oil, hyacinth abs., jasmin abs., juniper berry oil, labdanum res., lavander oil, laurel leaf oil, lavender oil, lemon oil, lemongrass oil, lime oil, lovage oil, mace oil, mandarin oil, mimosa abs., myrrh abs., mustard oil, narcissus abs., neroli bigarade oil, nutmeg oil, oakmoss abs., olibanum res., onion oil, opoponax res., orange oil, orange flower oil, origanum, orris concrete, pepper oil, peppermint oil, peru balsam, petitgrain oil, pine needle oil, rose abs., rose oil, rosemary oil, sandalwood oil, sage oil, spearmint oil, styrax oil, thyme oil, tolu balsam, tonka beans abs., tuberose abs., turpentine oil, vanilla beans abs., vetiver oil, violet leaf abs., ylang ylang oil and like vegetable oils, etc. Synthetic fragrance materials include but are not limited to pinene, limonene and like hydrocarbons; 3,3,5-trimethylcyclohexanol, linalool, geraniol, nerol, citronellol, menthol, borneol, borneyl methoxy cyclohexanol, benzyl alcohol, anise alcohol, cinnamyl alcohol, β-phenyl ethyl alcohol, cis-3-hexenol, terpineol and like alcohols; anethole, musk xylol, isoeugenol, methyl eugenol and like phenols; α-amylcinnamic aldehyde, anisaldehyde, n-butyl aldehyde, cumin aldehyde, cyclamen aldehyde, decanal, isobutyl aldehyde, hexyl aldehyde, heptyl aldehyde, n-nonyl aldehyde, nonadienol, citral, citronellal, hydroxycitronellal, benzaldehyde, methyl nonyl acetaldehyde, cinnamic aldehyde, dodecanol, α-hyxylcinnamic aldehyde, undecenal, heliotropin, vanillin, ethyl vanillin and like aldehydes; methyl amyl ketone, methyl β-naphthyl ketone, methyl nonyl ketone, musk ketone, diacetyl, acetyl propionyl, acetyl butyryl, carvone, menthone, camphor, acetophenone, p-methyl acetophenone, ionone, methyl ionone and like ketones; amyl butyrolactone, diphenyl oxide, methyl phenyl glycidate, .gamma.-nonyl lactone, coumarin, cineole, ethyl methyl phenyl glicydate and like lactones or oxides; methyl formate, isopropyl formate, linalyl formate, ethyl acetate, octyl acetate, methyl acetate, benzyl acetate, cinnamyl acetate, butyl propionate, isoamyl acetate, isopropyl isobutyrate, geranyl isovalerate, allyl capronate, butyl heptylate, octyl caprylate octyl, methyl heptynecarboxylate, methine octynecarboxylate, isoamyl caprylate, methyl laurate, ethyl myristate, methyl myristate, ethyl benzoate, benzyl benzoate, methylcarbinylphenyl acetate, isobutyl phenylacetate, methyl cinnamate, cinnamyl cinnamate, methyl salicylate, ethyl anisate, methyl anthranilate, ethyl pyruvate, ethyl α-butyl butylate, benzyl propionate, butyl acetate, butyl butyrate, p-tert-butylcyclohexyl acetate, cedryl acetate, citronellyl acetate, citronellyl formate, p-cresyl acetate, ethyl butyrate, ethyl caproate, ethyl cinnamate, ethyl phenylacetate, ethylene brassylate, geranyl acetate, geranyl formate, isoamyl salicylate, isoamyl isovalerate, isobornyl acetate, linalyl acetate, methyl anthranilate, methyl dihydrojasmonate, nopyl acetate, β-phenylethyl acetate, trichloromethylphenyl carbinyl acetate, terpinyl acetate, vetiveryl acetate and like esters, and the like. Suitable fragrance mixtures may produce a number of overall fragrance type perceptions including but not limited to, fruity, musk, floral, herbaceous (including mint), edible, and woody, or perceptions that are in-between (fruity-floral for example). Typically these fragrance mixtures are compounded by mixing a variety of these active fragrance materials along with various solvents to adjust cost, evaporation rates, hedonics and intensity of perception. Well known in the fragrance industry is to dilute essential fragrance oil blends (natural and/or synthetic) with solvents such as ethanol, isopropanol, hydrocarbons, acetone, glycols, glycol ethers, water, and combinations thereof, and using solvent up to as much as 90% of the volatizable fragrance composition. The preferred fragrance oil for use in the salt crystal air freshener of the present invention may be comprised of a mixture of many fragrance actives and volatile solvents, sometimes along with smaller amounts of emulsifiers, stabilizers, wetting agents and preservatives. More often than not, the compositions of the fragrance oil purchased from the various fragrance supply houses remain proprietary and thus can only be described in general terms. The fragrance oil is preferably incorporated at a level of from about 80% to about 99.8% by weight in the fragrance premix, and from about 0.8% to about 16% by weight of the total weight of the finished fragranced salt crystals.

Optional Adjuvant

The air freshener crystals of the present invention may also include dyes, pigments or other suitable colorants to provide aesthetic appeal to the fragranced crystals. Such dyes may include FD&C and/or D&C Yellows, Reds, Blues, Greens and Violets, or really any other dye or pigment, and such materials are commonly purchased in powder or liquid form. Dyes and/or pigments are incorporated at levels sufficient to provide light color to deep color to the finished crystals. When dyes or other pigments are desired for colored air freshener salt crystals, they are included in the fragrance premix at from about 0.0001% to about 1% by weight, depending on the concentration of the colorants (e.g. if a liquid/diluted dye or a neat powder).

The fragranced salt crystal air freshener of the present invention may also include one or more preservatives to help prevent dye fading and mold and other microbial growth on the crystals. The preferred microbial preservatives include Neolone® and Kathon® products from Lonza and Rohm & Haas. These materials are incorporated at the manufacturers' recommended levels in the fragrance premix to discourage bacterial and mold growth on the finished crystals. An ultraviolet inhibitor and/or an antioxidant may also be added to the fragrance premix to reduce dye fade on the finished crystals.

The present invention may also include one or more solvents to aid in control of viscosity of the fumed silica/emulsifier/fragrance oil premix. Some of these solvent materials overlap with what was defined as nonionic emulsifiers above, and include, but are not limited to, ethanol, methanol, isopropanol, n-propanol, n-butanol, MP-Diol (methylpropanediol), ethylene glycol, propylene glycol, and various glycol ethers (e.g. ethylene or diethylene glycol monoalkyl ethers, and propylene or dipropylene glycol monalkyl ethers, and the like), and mixtures thereof. These solvents may be used in any combination at a level of from about 0.1% to about 5% by weight in the premix to adjust viscosity such that the fragrance premix coats the salt crystals evenly. As mentioned, any of these solvents may be present in the proprietary fragrance oil as obtained from any number of fragrance suppliers, and it is possible to have the fragrance oil supplier add the viscosity reducing solvent to the purchased fragrance oil rather than for the end formulator to put it into the fragrance premix.

The Processing Methods

The preferred method for producing the fragranced crystals of the present invention involves a first step of producing a fragrance “premix” or “pre-blend”. The pre-blend preferably comprises from about 80% to about 99.8% by weight fragrance oil; from about 0.1% to about 10% by weight fumed silica; and, from about 0.1% to about 10% by weight nonionic emulsifier, along with optional dyes and other adjuvant. Most preferred is to prepare a premix comprising from about 90% to about 99% by weight fragrance oil; from about 0.1% to about 5% by weight fumed silica; and, from about 0.1% to about 5% by weight emulsifier, along with optional dyes and other adjuvant. When dyes or other pigments arc desired, they are included in the pre-blend at from about 0.0001% to about 1% by weight, depending on the concentration of the colorants (e.g. if a liquid/diluted dye or a neat powder). The fragrance oil, fumed silica and the optional dyes are first mixed together to form a wet silica paste. The emulsifier is then added and mixed thoroughly to produce a thickened liquid fragrance premix that is used to coat the salt crystals. Thicker pre-blends may be made by increasing the weight percent of fumed silica in the pre-blend. As mentioned, solvents such as alcohols may be added to thin the premix.

The second step for producing the fragranced crystals of the present invention is to use the above described premix to coat the salt crystals. Preferably a mixture is prepared in a tumbling blender comprising from about 80% to about 99% by weight of salt crystals and from about 1% to about 20% by weight of the above described pre-blend. Most preferred is to blend 95% to about 99% salt crystals by weight with from about 1% to about 5% by weight fragrance pre-mix. As mentioned, the preferred salt crystals are evaporative-sourced, and most preferably comprise natural sea salt crystals sieved to fall between about a ⅜ inch U.S. Standard sieve and about a 1 inch U.S. Standard sieve. A “V-blender”, rolling drum blender, “double-cone” blender or other suitable tumble-coating machinery is charged with the appropriate amount of salt crystals for the batch. The pre-blend is then added to the salt crystals and the mixer is turned on. The crystals are tumbled with the fragrance pre-mix until there is even coating of the crystals without excessive breakage and crushing into fines. It has been found that the “V-blender” and the “double cone” blender provide the best coating of the salt crystals without concomitant breakage of crystals.

Given the preferred weight percentages in the pre-blend and the preferred weight percentages of the pre-blend/crystals blending, it follows that the preferred weight percentages of the final air freshener crystals comprise: from about 80% to about 99% salt crystals; from about 0.8% to about 16% fragrance oil; from about 0.001% to about 2% fumed silica; and, from about 0.001% to about 2% emulsifier. It is most preferred that the final fragranced salt crystals of the present invention comprise: from about 95% to about 99% by weight natural sea salt crystals; from about 1% to about 5% by weight fragrance oil; from about 0.05% to about 0.15% fumed silica; and, from about 0.005% to about 0.015% emulsifier.

Compositions of the present invention are included in TABLE 1. The final fragranced crystals shown were tested along side two other retail air fresheners in consumer sensory evaluations. That comparative sensory data is shown in TABLE 2.

TABLE 1
Fragrance Premix and Finished Fragranced Salt Crystals

| | Weight % | |
|---|---|---|
| Component | Fragrance Premix | Fragranced crystals³ |
| Natural sea salt crystals¹ | — | 97.000 |
| Fragrance oil | 96.5 | 2.895 |
| Fumed Silica | 3.0 | 0.090 |
| Emulsifier² | 0.3 | 0.009 |
| Optional Dyes | 0.2 | 0.006 |

Notes:
¹Atlantic, Caribbean or Pacific Ocean natural solar salt crystals with distribution from about ⅜ inch to about 1 inch obtained by U.S. Standard sieves;
²Tween ® 80, Tween ® 60 or Tween ® 20, or mixtures thereof;
³Final composition based on blending 3 wt. % fragrance premix with 97 wt. % solar salt crystals.

TABLE 2
Fragrance Perception of Fragranced Salt Crystals:

| | T = 0 | 1 wk | 2 wk | 4 wk | 5 wk | 6 wk |
|---|---|---|---|---|---|---|
| | N = | | | | | |
| Description | 40 | 46 | 36 | 40 | 36 | 42 |
| 3/16″- 5/16″ Sieve range¹; Lavender fragrance² | 4.0 d | 3.9 c | 4.1 b | 3.9 c | 3.8 b | 3.6 b |
| 5/16″-⅜″ Sieve range; Lavender fragrance | 5.2 b | 4.4 b | 3.9 b | 4.4 b | 4.2 a | 4.0 a |
| ⅜″-⅞″ Sieve range; Lavender fragrance | 5.6 a | 4.9 a | 4.6 a | 4.8 a | 4.3 a | 3.8 ab |
| Retail Air Freshener (solid gel type); Lavender | 4.6 c | 3.5 d | 2.7 c | 1.7 d | 1.4 c | 1.4 c |
| Retail Air Freshener (passive liquid); Lavender | 4.0 d | 3.5 d | 3.8 b | 3.6 c | 3.5 b | 3.5 b |

Notes:
¹Sea salt crystals were previously sieved to the indicated ranges prior to coating with the fragrance premix.
²4% Fragrance premix to 96% salt crystals.
(3) Numerical entries are based on a 1-9 perception of fragrance strength by N-panelists brought into a small test room, where 1-extremely weak and 9-extremely strong fragrance perception.

The consumer test as summarized in TABLE 2 used sea salt crystals of three different sieve size distributions, with each of the distributions treated with the same level of identical fragrance premix. That is, the only difference between the first three entries in TABLE 2 is the crystal size. The retail air freshener products were chosen on the basis of having a similar fragrance, in this case a “lavender” (herbal/floral-type) fragrance. Over the course of 6-weeks, the fragrance crystals outperformed the passive gel and passive liquid air fresheners. Smaller crystal size was shown to give less fragrance delivery than larger crystal size distributions. Thus, the range of sieve size from about ⅜ inch to about 1 inch was optimal. Not being bound by any particular theory, it is believed the larger crystal size allows for greater air flow and fragrance release when the crystals are placed in an open jar.

We have thus described a unique and new invention that comprises fragranced salt crystals comprising salt crystals grown from evaporation of brine solution and coated with an emulsified fragrance premix. The fragranced salt crystals in accordance with the present invention will find use as an air freshener and may be merchandised separately (e.g. in a flexible sealed package such as a bag), with or without a decorative jar that may be filled with the crystals and placed in the home environment.

We claim:

1. An air freshener comprising:
a. salt crystals grown from evaporation of a brine solution, said crystals sieved to distribute between about ⅜ inch and about 1 inch U.S. Standard sieves;
b. fumed silica;
c. a nonionic emulsifier; and,
d. fragrance oil.

2. The composition of claim 1, wherein said salt crystals are sea or lake salt crystals, with said brine solution comprising ocean sea water or Great Salt Lake water.

3. The composition of claim 1, wherein said nonionic emulsifier is selected from the group consisting of sorbitan esters, alkoxylated sorbitan esters, C₂-C₆ glycols, glycol esters, glycerin, glyceryl esters, alkoxylated glyceryl esters, amide waxes, fatty alcohols, monoalcohol esters, polyethylene glycol, polypropylene glycol, polyethylene glycol esters, polypropylene glycol esters, fatty alcohol alkoxylates, alkyl phenol alkoxylates, alkoxylated fatty acid esters, alkanolamides, amine N-oxides, and alkylpolyglycosides, and mixtures thereof.

4. The composition of claim 1, wherein said fumed silica is untreated hydrophilic silicon dioxide of sub-micron size.

5. The composition of claim 3, wherein said nonionic emulsifier is an alkoxylated sorbitan ester.

6. The composition of claim 5, wherein said alkoxylated sorbitan ester is selected from the group consisting of ethoxylated sorbitan monooleate, ethoxylated sorbitan monolaurate, ethoxylated sorbitan monopalmitate, and ethoxylated sorbitan monostearate, and mixtures thereof.

7. The composition of claim 1, further comprising a solvent selected from the group consisting of ethanol, methanol, isopropanol, n-propanol, methylpropanediol, ethylene glycol, propylene glycol, diethylene glycol monoalkyl ether, and dipropylene glycol monoalkyl ether, and mixtures thereof.

8. The composition of claim 1, further comprising a preservative selected from the group consisting of antioxidants, uv absorbers, and antimicrobials, and mixtures thereof.

9. The composition of claim 1, further comprising a colorant.

10. A method of preparing the air freshener of claim 1, said method comprising the steps of:
a. mixing a fumed, untreated and hydrophilic silica, a nonionic emulsifier and a fragrance oil to form a premix;
b. sourcing salt crystals grown from the evaporation of a brine solution;
c. sieving said salt crystals across 1 inch and ⅜ inch Standard U.S. Sieves; and,
d. tumbling said premix with said sieved salt crystals until said crystals are evenly coated with said premix.
